(19) United States
(12) Patent Application Publication
(10) Pub. No.: US 2017/0181737 A1
(43) Pub. Date: Jun. 29, 2017

(54) METHOD AND APPARATUS FOR PASSING SUTURE THROUGH TISSUE

(71) Applicant: TENSOR SURGICAL, INC., Chattanooga, TN (US)

(72) Inventors: Brett Sanders, Signal Mountain, TN (US); Keith J. Harper, Chattanooga, TN (US)

(73) Assignee: TENSOR SURGICAL, INC., Chattanooga, TN (US)

(21) Appl. No.: 15/312,710

(22) PCT Filed: May 20, 2015
(86) PCT No.: PCT/US15/31674

§ 371 Date: Nov. 21, 2016

Related U.S. Application Data
(60) Provisional application No. 62/001,696, filed on May 22, 2014.

(51) Int. Cl. A61B 17/04; A61B 17/29; A61B 17/06

(52) U.S. Cl. CPC A61B 17/04; A61B 17/0469; A61B 17/0483; A61B 17/06109; A61B 17/29; A61B 2017/06042; A61B 2017/06009

(57) ABSTRACT

A suture grasping and passing device having a handle with an elongate tubular needle defining a lumen therein and extending from the distal end of the handle and having a pointed forward distal tip for puncturing soft tissue. A lateral radially open suture capture notch is provided in the tubular needle adjacent the pointed distal tip and defines a mouth open to the needle lumen. The notch mouth is contoured to receive a suture therein transversely across the mouth and an elongate gate member is slidably received for axial movement within the lumen whereby the gate member may be advanced in a forward direction to entirely close off an outer radial apex portion of the suture capture mouth to capture a suture under the gate member within the mouth of the notch.

### CROSS REFERENCE

This application claims the benefit of U.S. Provisional Application No. 62/001,696, filed on May 22, 2014, for RETROGRADE SUTURE GRASPING AND PASSING DEVICE, the contents of which are incorporated herein in its entirety.

### BACKGROUND OF THE INVENTION

The present invention relates to surgical apparatus and procedures, and more particularly to surgical apparatus and procedures for passing suture through tissue.

In endoscopic surgical procedures it is often necessary to pass suture through tissue. This is accomplished through the use of suture passer devices that manipulate, pass and retrieve suture through tissue, which can be challenging in an endoscopic or arthroscopic procedure where visualization and space is limited.

Various such suture passer devices have been developed. In some instances, retrieving suture is accomplished by simply grasping it with regular forceps or other graspers having a suture capturing aperture provided at the distal tip when the forceps jaws are closed. Other suture passer devices include an elongate tubular needle with a lateral slot or opening adjacent the pointed tip of the needle and a flexible arm that is slidably received within the lumen of the needle for grasping or trapping suture at the distal end of the needle. Examples of this latter described type of suture passer device follow.

U.S. Patent Application Publication No. 2014/0222033 to Foerster et al., for example, discloses a suture manipulating and passing device which includes a hollow needle body with a lateral slot at its distal end which is open to the needle lumen. A manipulatable wire extends through the lumen of the needle and further extends from the lateral slot to pin or trap suture against the needle in the lateral slot.

U.S. Patent Application Publication No. 2015/0094739, to Norton et al. discloses a suture passer device having an elongate tubular needle with a lateral slot adjacent the tip of the needle, and a flexible arm that slidably extends through the lumen of the needle and exits the slot for grasping suture and pulling the suture back into the lateral slot where it is trapped against the needle.

U.S. Patent Application Publication No. 2015/0112368 to Stewart et al. discloses a suture passing device which includes an elongate tubular needle with a lateral notch, slot or window formed in the needle adjacent the tip of the needle which communicates with the lumen. As with the afore described devices, it also includes a clamping rod which is slidably received within the needle lumen, and at the distal end is bifurcated into a first arm and a second arm, with one of the first and second arms extending distally of the other of the first and second arms, and including a clamping surface. The distal end of the outwardly extending arm extends outwardly through the lateral slot or window, and similar to the other described devices may be retracted to pin or trap the suture within the window.

A shortcoming with such suturing devices is that they are relatively complex to operate and do not assure secure suture grasping and retention with every attempt.

### SUMMARY OF THE INVENTION

The suture grasping and passing device of the present invention includes a handle with an elongate needle defining a lumen therein and extending from the distal end of the handle and having a pointed forward distal tip for puncturing soft tissue. A radially open suture capture aperture or notch is provided in the tubular needle adjacent the pointed distal tip and defines a mouth which is open to the lumen of the tubular needle. The mouth is contoured to receive a suture therein transversely across the mouth. An elongate gate member is slidably received for axial movement within the lumen of the needle whereby the gate member may be advanced in a forward direction for thereby actually totally closing off an outer radial apex portion of the mouth of the suture capture notch in order to assure capture a suture thereunder in the mouth of the notch.

The elongate gate member is constructed typically of a flexible wire, such as Nitonal. In addition, the forward distal end of the tubular needle may be curved in order to facilitate suturing in different surgical sites.

The mouth of the suture capture notch may also be contour to have a rearward facing reverse curvature which provides a recess curvature in the forward edge of the mouth for initially hooking and retaining a suture therein as the needle is being retracted.

In addition, the gate member is slidably engaged for axial movement within the lumen by manipulation of a thumb contact on the handle. A releasable lock mechanism may be provided on the handle for selectively locking the elongate gate member in position relative to the needle.

An additional advantage of the suture grasping and passing device of the present invention is that the gate member includes a finger protrusion extending forward in the axial direction from a circumferential edge of the distal end of the gate member. This finger protrusion is oriented within the lumen to slidably move therein in line with the apex of the notch mouth whereby the finger protrusion may be moved forward to completely close off the mouth with suture received therein and thereunder. The gate member is slidable to different selected positions whereby the finger protrusion may be selectively moved forward to literally clamp a suture in the notch mouth against the distal end of the gate member, or in the alternative, slid to a position to close off the suture capture mouth but permit desired lateral slippage of the suture which is captured in the mouth underneath the finger protrusion. Alternatively, the gate member may be slid sufficiently rearward within the needle lumen to thereby open the notch mouth for permitting ingress or egress of a suture in and from the mouth.

Unlike the suture grasping and passing devices of the prior art, the capture of suture within the mouth of the suture capturing notch is absolutely assured on every attempt as the device of the present invention literally closes off the mouth of the suture capturing notch with the suture retained therein, whereas the suture grasping and passing devices of the prior art rely upon a flexible arm to hopefully pin or trap the suture thereunder against the needle. In addition, the suture grasping and passing devices of the prior art do not provide alternative suture grasping techniques wherein the suture is alternatively positively clamped within the suture capture notch, or alternatively positively closes off the suture capture notch but permits lateral slippage of the suture across and within the suture capture notch.

### BRIEF DESCRIPTION OF THE DRAWINGS

Other objects and advantages appear hereinafter in the following description and claims. The accompanying drawings show, for the purpose of exemplification, without limiting the scope of the invention or the appended claims, certain practical embodiments of the present invention wherein:

FIG. 1 is a perspective schematic view of a first embodiment of the suture passer of the present invention with the proximal end of the handle cut from the figure:

FIG. 2 is an enlarged view of the forward needle tip of the suture passer shown in FIG. 1 without the inclusion of a suture;

FIG. 3 illustrates the suture passer distal tip shown in FIG. 2 with the internal gate member slidably advanced forward to close off the outer radial apex portion of the suture capture notch with the suture shown to be captured thereunder within the mouth of the notch;

FIG. 4 is an enlarged view of the forward distal end of the suture passer shown in FIG. 1 with a curved distal tip attached to the suture passer needle in lieu of the straight tip illustrated in FIG. 1;

FIG. 5 is a schematic view in side elevation illustrating another embodiment of the suture passer of the present invention being utilized for passing suture through tissue in an antegrade and a retrograde manner;

FIG. 6 is an enlarged perspective view of the forward end of the suture passer illustrated in FIG. 5 illustrated in an exploded configuration to show the elongate gate member, which is normally received within the lumen of the suture passer needle, positioned above the suture passer;

FIG. 7 is a perspective view of the suture passer shown in FIG. 6 as seen from a reverse perspective;

FIG. 8 is an enlarged perspective view of the forward distal tip of the suture passer shown in FIGS. 5 through 7 illustrating the distal tip of the needle being positioned to capture a suture for a retrograde pass;

FIG. 9 illustrates the suture passer tip shown in FIG. 8 upon hooking a suture for capture within the suture needle laterally exposed notch;

FIG. 10 is a perspective view illustrating the suture passer distal tip of FIG. 9 with the gate member slidably advanced forward to close off the apex portion of the suture passer suture capture notch with the suture captured thereunder;

FIG. 11 is an enlarged perspective view of the distal end of the suture passer hollow needle shown in FIG. 10 illustrating the interior parts thereof in dashed outline; and

FIG. 12 is a reverse perspective view of the suture passer distal end illustrated in FIG. 11.

### DETAILED DESCRIPTION OF THE PREFERRED EMBODIMENTS

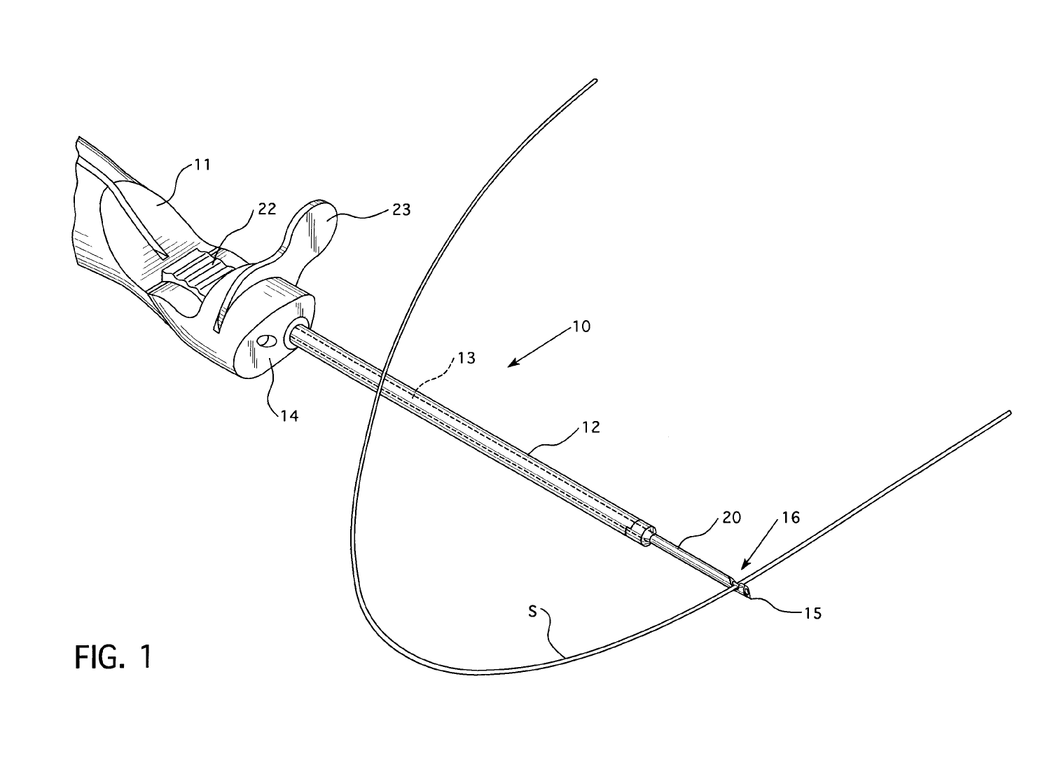

Referring to FIGS. 1 through 4, the suture grasping and passing device 10 of the present invention includes a handle 11 with an elongate tubular needle 12 defining a lumen 13 therein with the needle 12 extending from the distal end 14 of the handle 11. The tubular needle 12 is provided with a pointed forward distal tip 15 for puncturing soft tissue.

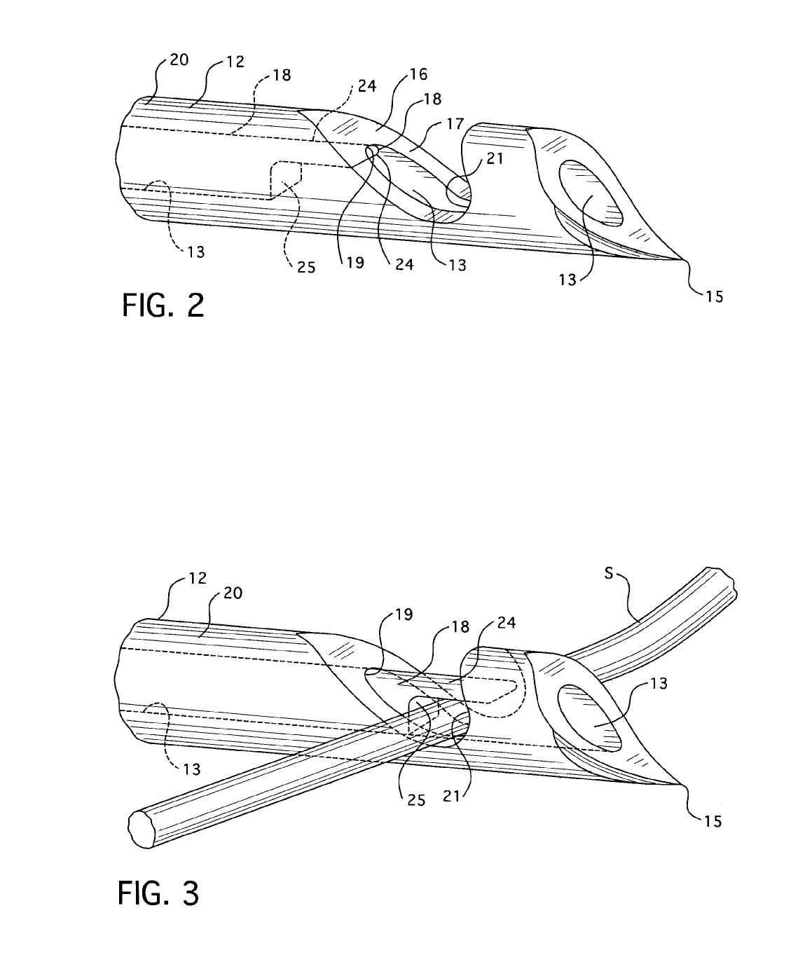

A laterally exposed and radially open suture capture notch 16 is provided in tubular needle 12 adjacent pointed distal tip 15. Notch 16 defines a mouth 17 open to lumen 13 of tubular needle 12. Mouth 17 is contoured to receive a suture S therein transversely across mouth 17 as illustrated in FIG. 3. Elongate gate member 18 is slidably received for axial movement within lumen 13 whereby gate member 18 may be advanced in a forward direction for thereby fully closing off an outer radial apex portion 19 of mouth 17 to provide assured capture of suture S thereunder in mouth 17 as illustrated in FIG. 3.

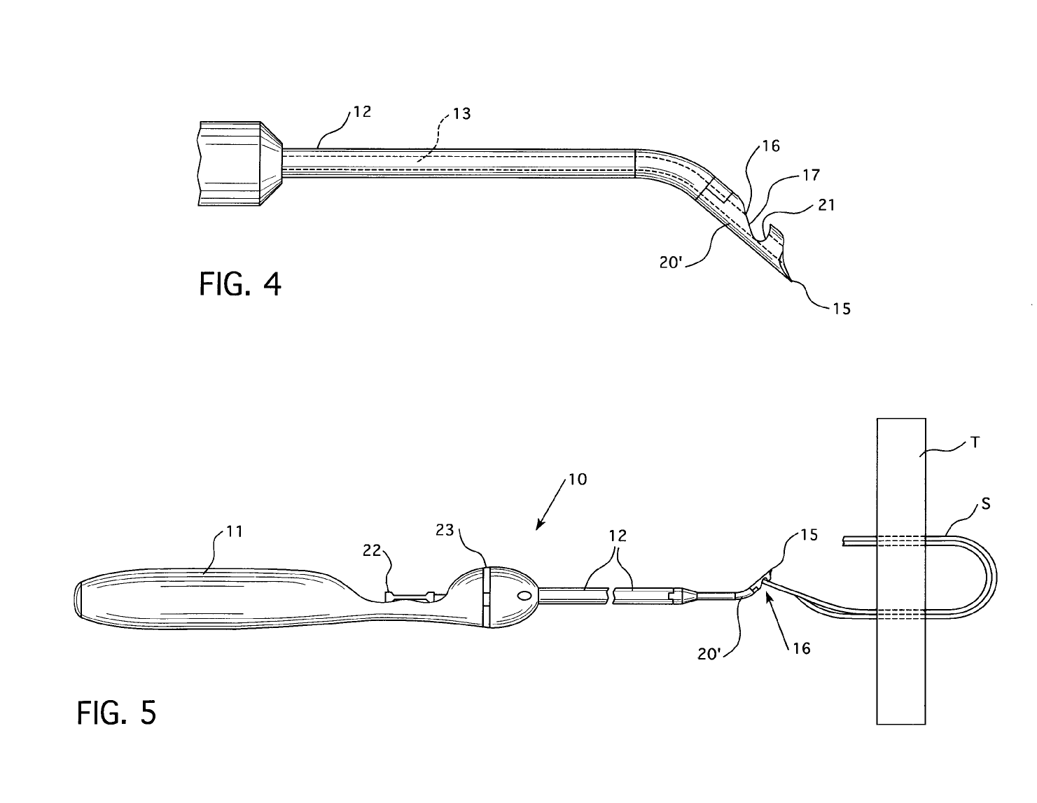

Elongate member 18 is constructed of Nitinol or similar flexible wire which has flexibility, if required, to follow a curved needle pass. As illustrated in FIG. 4, the straight distal end 20 of tubular needle 12 may be substituted with a curved distal end 20′. Mouth 17 of suture capture notch 16 is contoured to have a rearward facing reverse curvature providing a recessed curvature 21 in the forward edge of mouth 17 for hooking and retaining suture S therein as the needle 12 is retracted, thereby providing easy capture of suture S for retrograde suture passage.

Gate member 18 is slidably engaged for axial movement within lumen 13 by slidable manipulation of thumb contact 22 on handle 11 in a forward and rearward manner. A releaseable lock 23 is pivotally secured to handle 11 and may be thumb manipulated for selectively locking elongate gate member 18 in position relative to tubular needle 12.

Gate member 18 includes a finger protrusion 24 extending forward in the axial direction from a circumferential edge of distal end 25 of gate member 18. Finger protrusion 24 is oriented within lumen 13 to slidably move therein in line with the apex portion 19 of notch mouth 17 whereby finger protrusion 24 may be moved forward to completely close off mouth 17 with suture S received therein as illustrated in FIG. 3.

Gate member 18 is slidable to different selected positions whereby finger protrusion 24 may be selectively moved forward to clamp a suture in mouth 17 against the distal end 25 of gate member 18, or fully retain a suture in mouth 17, but permit desired lateral slippage of the suture S captured in mouth 17 as illustrated in FIG. 3. Additionally finger protrusion 24 may be positioned to fully open mouth 17 as illustrated in FIG. 2 for permitting ingress or egress of suture S in and from mouth 17.

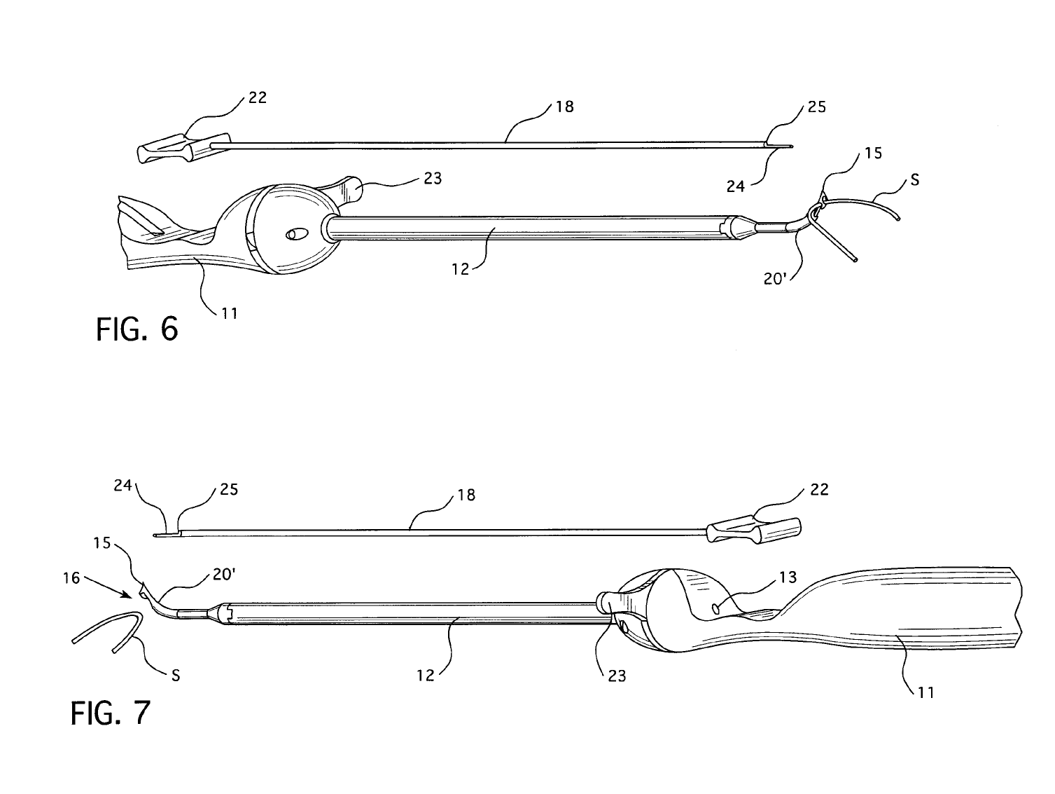

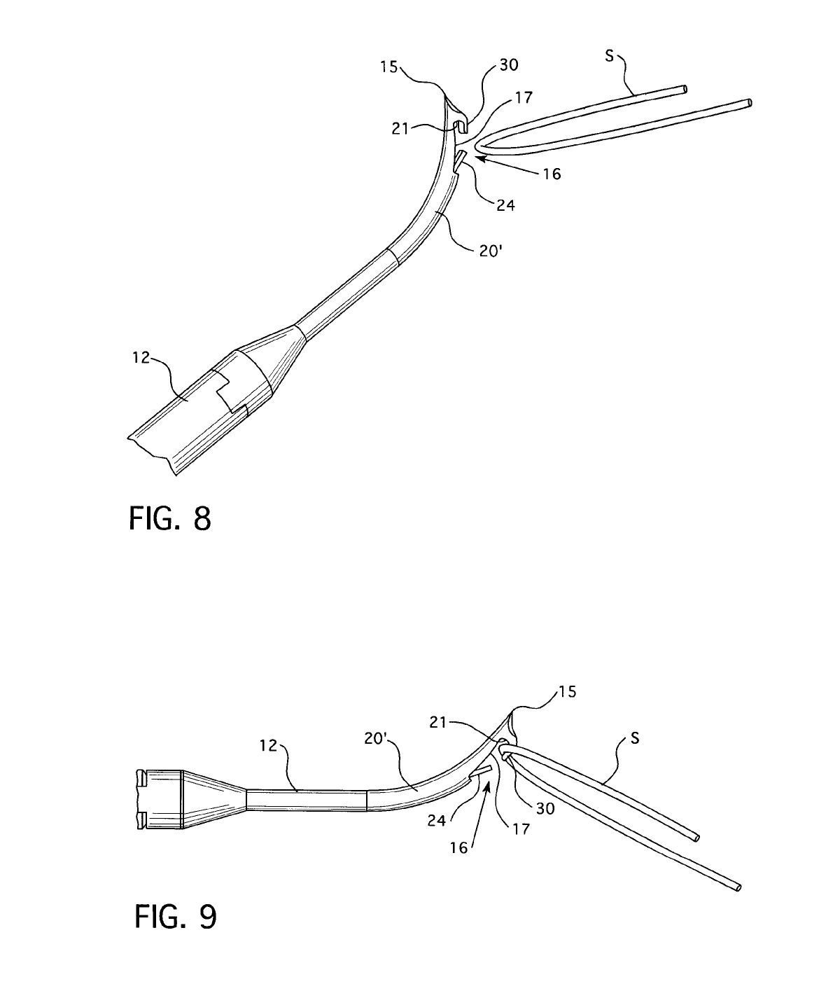

Referring next to the embodiment illustrated in FIGS. 5 through 11, the suture passer embodiment herein illustrated is in all respects similar in operation to the embodiment previously described, and accordingly, identical or similar parts are illustrated with the same reference numerals. The principal difference in this embodiment lies in the manner in which the elongate gate member slidably closes off the outer apex portion 19 of mouth 17.

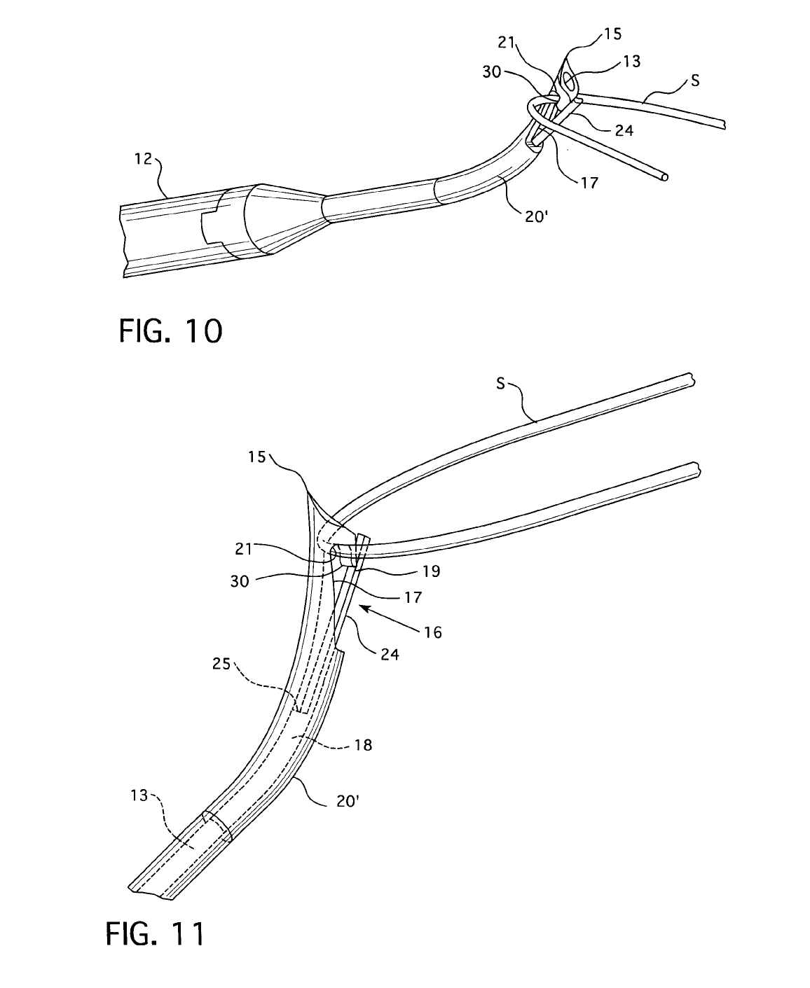

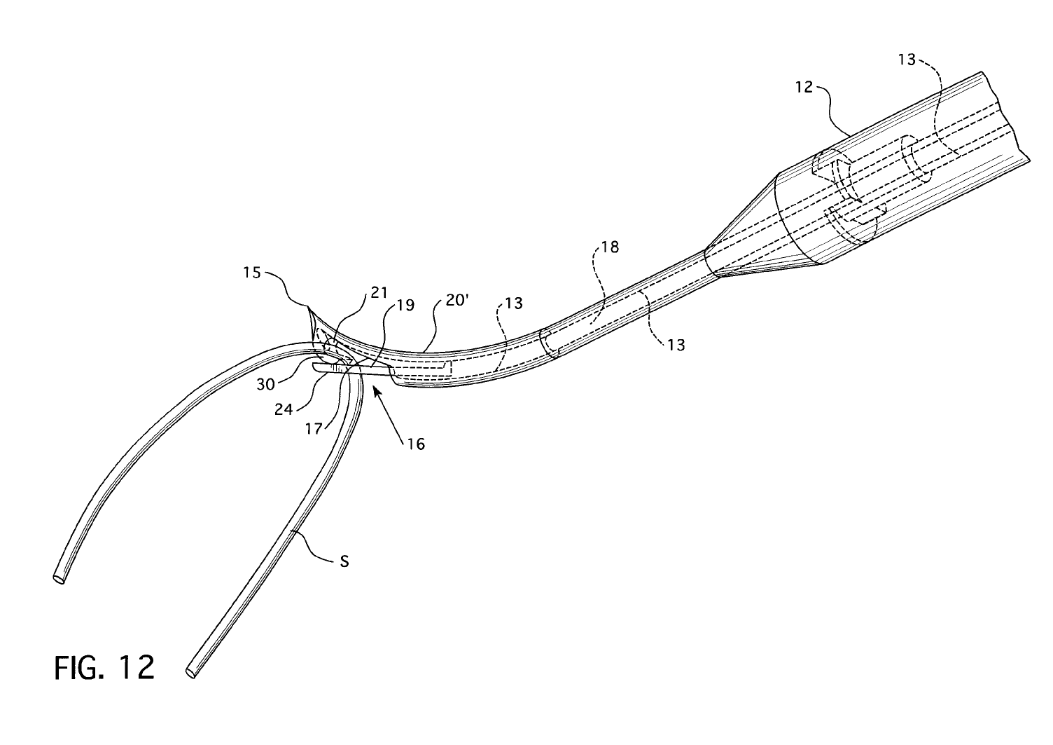

In this embodiment, the finger protrusion 24 may be slid forward in the same manner to entirely close off apex portion 19 of mouth 17, however, in this embodiment, finger protrusion 24 engages and slides over the outer face of distal hook tip 30 as illustrated in FIGS. 10, 11 and 12, instead of inside of hook distal tip 30 as illustrated in the previous embodiment. This embodiment securely and entirely closes off the apex portion 19 of mouth 17, but the distal tip of finger protrusion 24 cannot incidentally or accidentally engage and pinch suture S within the recess 21 of mouth 17. In all other respects, operation of the two embodiments illustrated are identical.

The suture grasping and passing device 10 of the present invention may be used in all types of soft tissue repair, but is especially adaptable to rotator cuff repair when one wishes to pull a suture which is already underneath the cuff through the rotator cuff in a retrograde manner after piercing the cuff from above at the desired point of suture passage with the needle 12. The device 10 of the present invention may also be used in the same fashion for labrum or other soft tissue repairs in the shoulder, hip or other joints. The device 10 also allows for antigrade passage of the suture by capturing the suture in the mouth 17 of needle 12 before the needle is pressed through the soft tissue T. This allows the suture to be pushed through the soft tissue T then released and left in the soft tissue after the needle is removed. Repeated antigrade and retrograde cycles with the same suture at different points in the cuff allow for a stitching of the soft tissue T as illustrated schematically in FIG. 5.

We claim:

1. A suture grasping and passing device comprising:
a handle;
an elongate tubular needle defining a lumen therein and extending from a distal end of the handle and having a pointed forward distal tip for puncturing soft tissue;
a radially open suture capture notch in said tubular needle adjacent said pointed distal tip and defining a mouth open to the lumen of said tubular needle and contoured to receive a suture therein transversely across said mouth; and
an elongate gate member slidably received for axial movement within said lumen of said needle whereby said gate member may be advanced in a forward direction for thereby closing off an outer radial apex portion of said mouth to capture a suture thereunder in said mouth.

2. The suture grasping and passing device of claim 1, wherein said elongate gate member is a flexible wire.

3. The suture grasping and passing device of claim 2, wherein the forward distal end of said tubular needle is curved.

4. The suture grasping and passing device of claim 1, wherein said mouth of said notch is contoured to have a rearward facing reverse curvature providing a recessed curvature in the forward edge of said mouth for hooking and retaining a suture therein as said needle is retracted.

5. The suture grasping and passing device of claim 1, wherein said gate member is slidably engaged for axial movement in said lumen by manipulation of a thumb contact on said handle.

6. The suture grasping and passing device of claim 1, including a releaseable lock on said handle for selectively locking said elongate gate member in position relative to said needle.

7. The suture grasping and passing device of claim 1, said gate member including a finger protrusion extending forward in the axial direction from a circumferential edge of the distal end of said gate member, said finger protrusion oriented in said lumen to slidably move therein in line with said apex of said notch mouth whereby said finger protrusion may be moved forward to close off said mouth with suture received therein.

8. The suture grasping and passing device of claim 7, wherein said gate member is slidable to different selected positions whereby said finger protrusion may be selectively moved forward to clamp a suture in said mouth against said distal end of said gate member, or retain a suture in said mouth but permit lateral slippage of the suture captured in said mouth, or open said mouth for permitting ingress or egress of a suture in said mouth.

9. A method of endoscopically manipulating and passing suture comprising the steps of:
providing an instrument comprising an elongate tubular needle having a pointed distal tip at its forward end and a radially open suture capture notch in said tubular needle adjacent to said pointed tip, and further comprising an elongate gate member slidably received for axial movement within said tubular needle and said gate member is oriented and slidably advanceable within said tubular needle to close off an outer radial apex portion of said notch;
positioning a suture laterally in and across said notch;
capturing said suture in said notch by advancing said gate finger protrusion to close off said notch and thereby capture said suture therein.

10. The method of claim 9, wherein said gate member is further provided with a finger protrusion extending forward in the axial direction from a circumferential edge of the distal end of said gate member and said finger protrusion is advanced sufficiently to clamp said suture in said notch against said distal end of said gate member.

11. The method of claim 9, further comprising piercing tissue at a first location with said needle to place the needle through the tissue and drawing the suture through the tissue;
retracting said gate member to open said notch and displacing said suture from said notch; and
removing said needle from said tissue, leaving said suture placed therein.

12. The method of claim 11, prior to positioning suture into said notch, piercing said tissue at a second location with said needle; then
positioning said suture in said notch;
capturing said suture in said notch by advancing said gate member to close off said notch and thereby capturing said suture therein; and
drawing said suture back through said second location in said tissue.
